(19) United States
(12) Patent Application Publication
(10) Pub. No.: US 2004/0028720 A1
(43) Pub. Date: Feb. 12, 2004

(54) Article for debridement & detoxification of wounds

(75) Inventors: John B. McAdams, Deerfield Beach, FL (US); Graydon Vadas, Charleston, SC (US); Michael A. Warshaw, Savannah, GA (US); Charley Hendrix, Savannah, GA (US)

Correspondence Address: AKERMAN SENTERFITT, P.O. BOX 3188, WEST PALM BEACH, FL, 33402-3188, US

(73) Assignee: Remedy Marketing, Inc., Charleston, SC

(21) Appl. No.: 10/342,082

(22) Filed: Jan. 14, 2003

Related U.S. Application Data
(63) Continuation-in-part of application No. 09/940,007, filed on Aug. 27, 2001, now abandoned.
Application No. 09/940,007 is a continuation of application No. 09/222,111, filed on Dec. 29, 1998, now abandoned.

(51) Int. Cl. A61K 38/48; A61K 9/70; A61K 7/06; A61K 7/11

(52) U.S. Cl. 424/443; 424/94.63; 424/70.14

(57) ABSTRACT

An abrasive pad for delivering a skin treatment for the treatment of animal and marine stings. The pad includes a material having an abrasive and entangling surface, the surface being sufficiently abrasive to mechanically abrade at least the stratum corneum from a patient's skin and the surface adapted to entangle any stinging cells left in the sting. A treatment solution is applied to the pad for contact with the patient's skin. The treatment solution comprises a 0.1% to 20% solution of a proteolytic enzyme.

### CROSS-REFERENCE TO RELATED APPLICATIONS

This application is a continuation-in-part of U.S. patent application Ser. No. 09/940,007, filed Aug. 27, 2001, which is a continuation of U.S. patent application Ser. No. 09/222,111, filed Dec. 29, 1998.

### TECHNICAL FIELD

This invention relates to an article for the treatment of bites, stings or wounds caused by animals and insects such as fire ants, jellyfish, sea lice, related arthropods and cnidarians, as well as other biologically-caused wounds and envenomations.

### BACKGROUND OF THE INVENTION

Fire ants (Solenopsis Invicta), sea lice (one of 1000 species of cnidarians), and related arthropods and cnidarians and other species are prevalent in tropical and sub-tropical areas, and can cause painful bites and stings. Fire ants in particular behave very aggressively, and can inflict very painful and toxic stings. The fire ant stings have been known to be fatal to humans, particularly as S. Invicta releases an airborne chemical signal which triggers all nearby fire ants to begin to attack the same victim, causing multiple stings. The sting, which is extremely painful to the recipient, can form a raised pustule which can rupture and become infected, leading to scarring.

Sea lice, which are the larval stage of the thimble jellyfish (Linuche Unguiculata) and stings from other cnidarians (jellyfish) can also cause painful stings, which if left untreated can cause a rash and blistering of the affected area, due to nematocysts left in the wound which continue to release toxins.

Current treatments for envenomation by fire ants and cnidarians are similar, and are typically topical measures such as the application of a cortisone cream, colloidal preparation or calamine solution. Vinegar, alcohol and meat tenderizer are also recommended. In more severe cases, antihistamines, steroids, and epinephrine may be administered by I.M., I.V., and/or orally.

There are a few instances noted in the literature of remedies for stings, particularly for jellyfish stings, and several noted in “Dangerous Marine Animals” by Dr. Bruce W. Halsted of the World Life Research Institute are of interest. Halstead on several occasions refers to using sand or adhesive tape to strip the wound site after a sting. There are also recommendations to use a razor with shaving cream to strip the outer layers of the wound for treatment. Alternative treatment recommendations include forming a mud or sand paste, and using a sharp edge such as the edge of a shell or a piece of wood to scrape the wound and thus remove nematocysts. Other recommendations include using tweezers, a towel, rag, seaweed or a stick to carefully remove nematocysts from wounds.

Physicians have long used dermabrasion to treat problems ranging from acne to burns, in order to speed up cell renewal and turnover. However, delivery systems for medicaments tend to involve deep delivery of medicaments, such as by hypodermic needle. For the treatment of toxins delivered by Solenopsis Invicta and other stinging animals, a shallow delivery system is needed.

### SUMMARY OF THE INVENTION

One aspect of the invention relates to an abrasive pad for delivering a skin treatment for the treatment of animal and marine stings. The pad comprises a material having an abrasive and entangling surface, with the surface sufficiently abrasive to mechanically abrade at least the stratum corneum from a patient's skin and the surface adapted to entangle at least one of a stinger and stinging cells left in the skin. A treatment solution is applied to the pad for contact with the patient's skin. The treatment solution includes a 0.1% to 20% solution of a proteolytic enzyme.

The treatment solution may be a 0.5% to 5% solution of a proteolytic enzyme, or may be a 2% solution of a proteolytic enzyme. The proteolytic enzyme may be papain.

The treatment solution may include a surfactant, which may be soap. In addition, or instead, the treatment solution may include an antitoxin, an antibiotic, an antifungal treatment, an antiseptic, an antibacterial agent and/or a soothing and healing agent. The antibacterial agent, if used, may be trichlosan. The soothing and healing agent, if used, may be aloe. The treatment solution may be a pH titer.

The pad may be multilayered. In one arrangement, the pad may include an absorbent material carrying said treatment solution, and a porous abrasive material. In another arrangement, the pad may include a burstable receptacle containing said treatment solution. The pad may include three layers including two outer layers and a central layer, at least one of the outer layers being of an abrasive material, and the burstable receptacle forming the central layer. A plurality of burstable receptacles may be provided, at least one of said burstable receptacles containing a different treatment solution from at least one other of said burstable receptacles.

The abrasive and entangling surface is formed of matted non-woven fibers. Alternatively, the abrasive and entangling surface is formed of a loop fabric, or may be sponge-like. The pad may in one arrangement be a mitt adapted to be worn over a hand of a user.

The sting may have been caused by an arthropod. The arthropod may be Solenopsis Invicta. Alternatively, the sting may have been caused by a cnidarian. The cnidarian may be a larval of the thimble jellyfish.

### BRIEF DESCRIPTION OF THE DRAWINGS

FIG. 1 illustrates the length of a typical Solenopsis Invicta stinger.

FIG. 2 illustrates an armed nematocyst.

FIG. 3 illustrates a discharged nematocyst.

FIG. 4 illustrates the average human epidermal layer of skin.

FIG. 5 is a side perspective view of a pad containing a solution according to the present invention.

### DETAILED DESCRIPTION

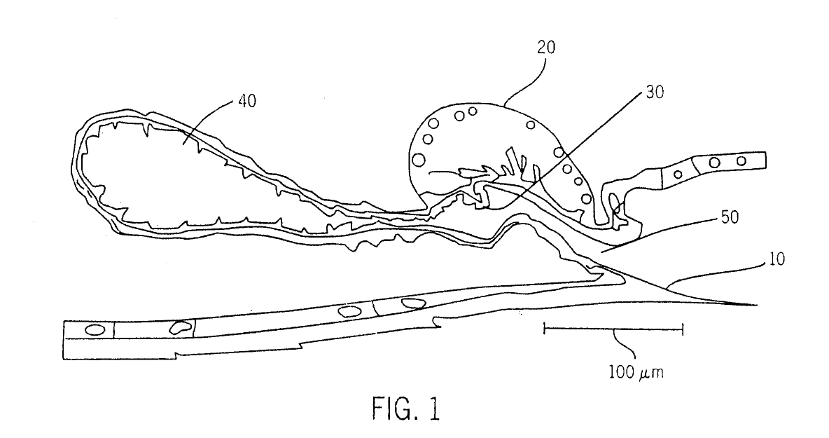

Solenopsis Invicta is a specific group of ants that are known for their aggressive behavior and their extremely painful and dangerous stings, which have been known to be fatal. A characteristic of Solenopsis Invicta and a number of other stinging arthropods is that their toxins, although powerful, are largely deposited in shallow wound sites, due to the small size of the stinger. As illustrated in FIG. 1, the stinger typically has a length of about 100 microns. The glandular epithelium 20 deposits or emits toxin 30 to a reservoir 40. The stinger 10 opens a hole or wound opening in the top layer of skin, the epidermis, of a recipient of about one-half to two-thirds of the length of the stinger and the toxin 30 enters the wound opening or hole via passage 50.

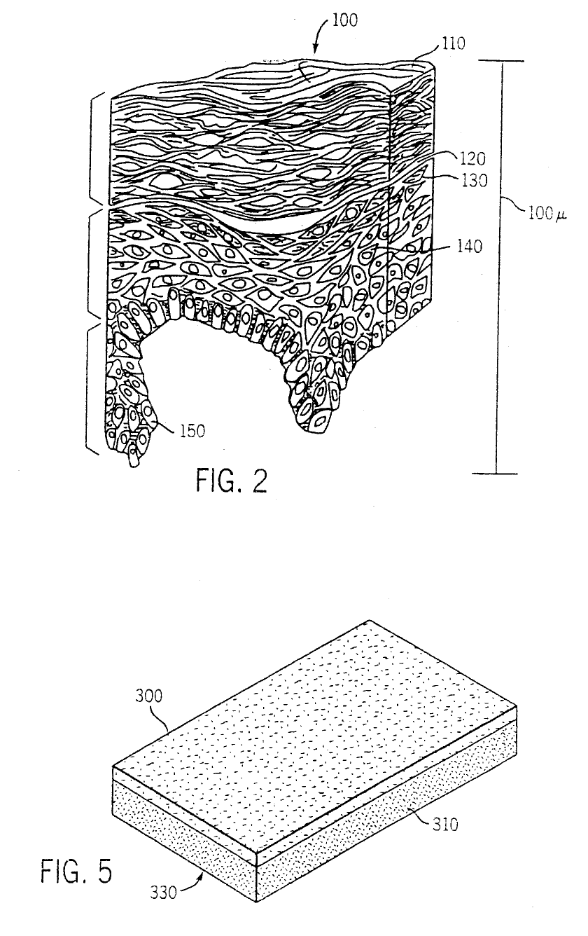

Besides arthropods, the invention is applicable for cnidarians and other biological envenomations (for example Coelenterates) stings. The cnidarians include hydras, men o'war, jellyfish, sea anemones, hydrodroids, corals, bryozoans and the like. A characteristic of cnidarians are nematocysts. These are the stinging cells which are usually deployed for food acquisition and/or for defensive purposes. Since all nematocyst-bearing cnidarians have essentially the same stinging cells, the nematocysts of prime concern are stinging and stinging/tangling. FIGS. 2 and 3 illustrate an armed nematocyst 200 and a discharged nematocyst 210 respectively.

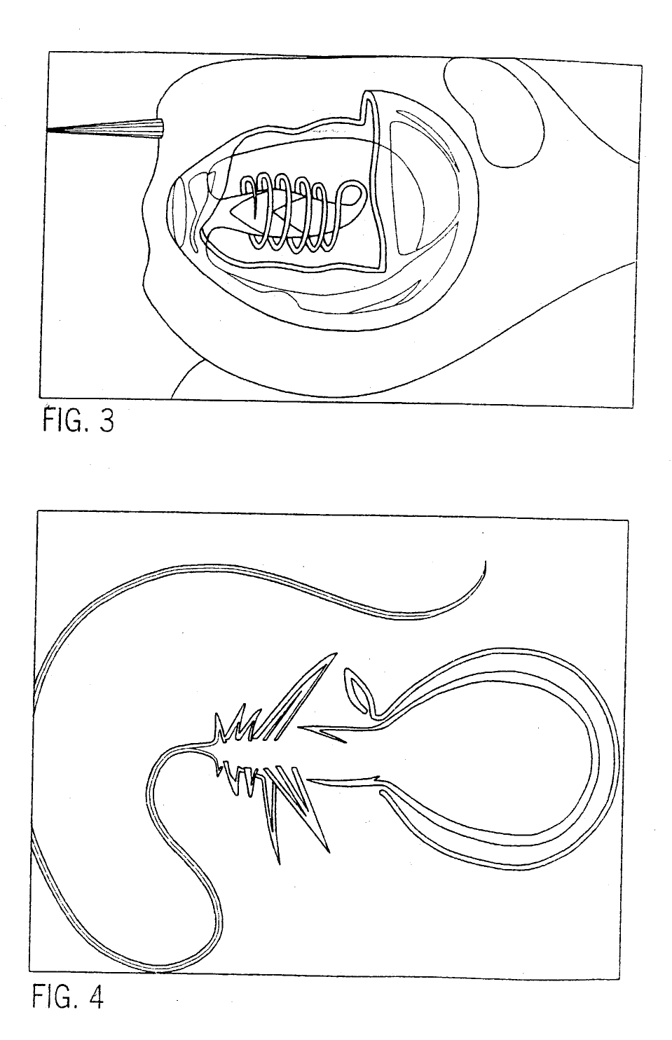

FIG. 4 illustrates the average human epidermis 100 having a typical thickness ranging from 70 to 120 microns or an average thickness of 100 microns. The epidermis 100 has five distinct layers. The first layer 110 is the stratum corneum. The second layer 120 is the stratum lucidum. The third layer 130 is the stratum granulosum. The forth layer 140 is the stratum spinosum. The fifth layer 150 is the stratum basale.

A delivery pad 300 is illustrated in FIG. 5, having a solution 310 saturated in the pad. The pad 300 includes an abrasive area, which may be present on one side 320, or may be present on second side 330. The sides 320 and 330 can differ from one another, for example, one side may be very abrasive, and the other side may be less abrasive. One of the sides may be smooth.

The pad 300 may be formed of a single layer of abrasive material. Alternatively, the pad 300 may be multilayered, with an absorbent material backing a porous abrasive material. The absorbent material can carry a solution of a treatment substance. The treatment substance can be delivered through the abrasive material on the application of slight pressure. The pad may be pre-saturated with a treatment solution, and may be stored in a sealed watertight container to prevent drying out of the pad during storage. An alternative arrangement (not shown) can include a bladder or other receptacle either alone or in combination with the abrasive and/or absorbent material. The application of slight pressure could be used to rupture the bladder or receptacle in order to release the treatment substance into the pad. The pad could take the form of a three-layer pad, with the three layers comprising an abrasive material on one side of the pad, an absorbent material on the other side of the pad, and the central layer being formed of the bladder. A plurality of bladders can be provided, with each bladder containing a different solution that may be mixed by rupturing the bladders, either in a predetermined sequence, or all at once.

The pad material may be of a construction including varying degrees of abrasiveness for the application for which it was designed. A pad with one side more abrasive than the other can be beneficial so that a first, more abrasive side may be used first, followed by a second, less abrasive side. The texture of the pad may be in an amorphous, unpatterned, construction which facilitates extraction of envenomating stinging cells such as nematocysts through entanglement.

The abrasive pad may be formed from matted or entangled non-woven fibers, or may be formed from a loop fabric similar to the loop portion of hook-and-loop fasteners (Velcro) and the like. Alternatively, the abrasive pad may contain a plurality of apertures, forming a sponge-like body. The apertures or areas between fibers of the pad may have a random size distribution, or may all be of the same size. The size of the apertures or areas between fibers of the pad are preferably of a size to enable at least part of the stinging cell to penetrate into the aperture when the pad is brought into contact with the wound, such that further movement of the pad causes the stinging cell to become entangled in the pad and hence removed from the wound with the pad. Multiple passages of the pad over the wound may entangle most or all of the stinging cells into the pad, thus removing the stinging cells from the wound.

The pad may be formed of a plastic material, or any other material of a fibrous or abrasive nature. The pad should be sufficiently abrasive so that surface layers of the skin may be removed with the pad.

The abrasive pad can be used to remove any remnants and pieces of stinging cells or tentacles still in or on the wound. The pad is flexible and may be used as a means for removing the tentacular remnants with their undischarged nematocysts. Typically at least the stratum corneum is removed or abraded along with the tentacular remnants and undischarged nematocysts. It will be appreciated that not all stings will leave a stinger or stinging cells in the wound, particularly if caused by fire ants and bees. However, the pad may still be used to clean the wound and remove at least the stratum corneum of the skin. The treatment solution may then be easily delivered into the remaining layers of the skin.

In one arrangement, the pad contains a treatment solution containing a proteolytic enzyme such as papain, bromelain, peptidase, protease, trypsin, chymo-trypsin and various combinations of these enzymes. It should be noted that proteolytic enzymes may be synthetically created and may thus have names which are not necessarily associated with the original enzyme from which they were synthesized. The solutions may range in strength from 0.1% to 20% by weight. The reason for the wide range is due to the differing degrees of toxicity caused by the different creatures. For example, a lesser jellyfish such as Aueralias, known as Moon Jelly, has a reduced toxicity, and a strength of 0.1% proteolytic enzyme solution may adequately neutralize the toxin in a wound. A more toxic jellyfish such as Chironex Fleckerii, the deadly Box Jellyfish of Australia may require a 20% proteolytic enzyme solution for effectiveness. A 0.5% to 5% range will generally treat most envenomations. A 20% solution is generally the upper range of strength found to be effective without causing injury to the patient, and with an established history of dermatological application. In certain circumstances, however, it may be appropriate to use a solution having an amount of proteolytic enzyme higher than 20%. It should be noted that a very high strength solution would generally require a physician's prescription, as it could burn young and sensitive skin.

In some embodiments, the proteolytic enzyme solution may be mixed with other substances. For example, an anti-bacterial agent such as Triclosan (registered trademark) may be incorporated into the treatment solution. A surfactant may be incorporated instead of or in addition to an anti-bacterial agent. A soothing and healing agent, such as Aloe, may be incorporated. Alcohol such as methanol may be included. In other embodiments, the other substances, such as soothing and healing agents, may be applied after the application of the proteolytic enzyme solution.

The instant invention was tested on approximately 100 individuals that had received stings of the type described above. The recipient took a similar pad as described above that was saturated with a solution of soap and sterile water and a 2% papain enzyme solution. The pad was kept sterile and moist in a foil package. The pad used was relatively stiff, and had an abrasive texture sufficient to plane the skin and deliver the solution to the planed epidermis. The method of the invention was used with success by over 95% of the participants, with immediate resolution of suffering by the recipient. The invention was also successful on bites or stings of unidentified species and genus.

Further testing showed the successful use of a pad and method according to the invention on the marine and land species identified in Table 1 below.

TABLE 1

| CLASSIFICATION | COMMON NAME | DISTRIBUTION |
|---|---|---|
| PORIFERA | | |
| Family Desmacidonae | Red Moss Sponge | U.S. Cape Cod to S. |
| Family Tendaniidae | Fire Sponge | Carolina, West Indies |
| COLENTERATA | | |
| Hydrozoans | | |
| Family Milliporidae | Stinging and/or Fire Coral | Tropical Pacific and Indian |
| | | Oceans, Red Sea, |
| | | Caribbean |
| Family Plumulariidae | Stinging Seaweed | Tropical Pacific and Indian |
| | | Oceans, West Indies |
| Family Physaliidae | Atlantic Portuguese | Atlantic Ocean, |
| | Man-O-War | Mediterranean Sea |
| | Indo-Pacific | Tropical Indian and Pacific |
| | Man-O-War | Oceans |
| SCYPHOZOA | | |
| Family Chirodropidae | Deadly Sea Wasp; Box | Australian Pacific Coast |
| | Jellyfish | |
| Family Carybdeidae | Sea Wasp | Tropical Pacific, Atlantic |
| | | and Indian Oceans. |
| Family Cyaneidae | Sea Blubber; Lion's Mane | Northern Atlantic & Pacific, |
| | | Baltic Sea, Tropical and |
| | | Temperate Pacific Ocean |
| ANTHOZOA | Sea Anemones & Coral | World-Wide |
| Family Acroporidae | Elkhorn Coral | Caribbean Sea |
| Family Actiniidae | Sea Anemone | Eastern Atlantic, |
| | | Mediterranean, Black Sea |
| | | and Sea of Asia |
| Family Actinodendronidae | Hell's Fire Sea Anemone | Atlantic and Pacific Oceans |
| Family Aliciidae | Sea Anemone | Red Sea |
| Family Hormathiidae | Sea Anemone | Eastern Atlantic, |
| | | Mediterranean Sea, Coastal |
| | | Africa |
| ANNELIDA | Segmented Worms | World-Wide |
| Family Amphinomidae | Fire Worm | Gulf of Mexico, Tropical |
| | | Pacific and Atlantic Oceans |
| ENCHINODERMATA | Starfish, Sea Cucumbers, | World-Wide |
| | Sea Urchins | |
| Class Holothuroidae | Sea Cucumber | All temperate bodies of |
| | | water |
| Family Toxopneustidae | Sea Urchin | Pacific and Indian Oceans |
| ECTOPROCTA | | |
| Family Alcyonidioidae | Bryozoans | World-Wide |
| ARTHROPODIA | Spiders and Insects | World-wide |
| Myrmicinae | Ants | World-Wide |
| Solenopsis | Fire Ants and other ants | World-Wide |
| Pogonomyrmex | | |
| Paraponera | | |
| Diptera and Acarina | No-see-ums, punkies, | World-Wide |
| Nematocera | gnats, moose flies, midges, | |
| Ceratopogonidae | biting midges, blood | |
| Culicoides | sucking gnats, mites, | |
| Acari | chigoe, jigger, chigger, | |
| Culex | fleas, blow flies, blue bottle, | |
| Trichoceridae | biting housefly, stable fly, | |
| Mycetophilidae | buffalo gnats, black flies | |
| Chaoborinae | | |
| Calliphoridae | | |
| Simuliidae | | |

In one embodiment, the pad may be formed into a mitt similar to an elbow length barbeque mitt. The mitt may be lined with an unpenetrable layer of plastic. The mitt may be similarly saturated with the previously prepared solution, for example, water, a cysteine proteolytic enzyme, and aloe. The mitt may be used to remove existing tentacle still on the patient, while protecting the wearer of the mitt from undischarged nematocysts. The mitt may be used to exfoliate and debride the wound, provide a reservoir of detoxifying solution and to provide a means of delivery of the detoxifying solution. The mitt may be especially useful for use with very toxic venom, such as that produced by the box jellyfish. Hereafter referred to as Chironex, the Box Jelly has nematocysts which can penetrate to a depth of 2-3 mm. Because of this depth of penetration and the deadly toxicity of the venom, extreme care must be taken in treating these victims to prevent the caregiver from being victimized by the same creature. The mitt may have the highest degree of abrasion that is medically acceptable. The mitt protects the person cleaning the wound from coming into contact with undischarged nematocysts. The mitt should be sufficiently supple to allow various areas of the human body to be treated with at least some degree of flexion of the mitt to treat in rounded and not readily accessible areas.

It should be understood that the examples and embodiments described herein are for illustrative purposes only and that various modifications or changes in light thereof will be obvious to persons skilled in the art, and that such modifications or changes are to be included within the spirit and purview of this application. Moreover, the invention can take other specific forms without departing from the spirit or essential attributes thereof.

What is claimed is:

1. An abrasive pad for delivering a skin treatment for the treatment of animal and marine stings, comprising:
a material having an abrasive and entangling surface, wherein the surface is sufficiently abrasive to mechanically abrade at least the stratum corneum from a patient's skin, wherein the surface is adapted to entangle at least one of a stinger and stinging cells left in the skin; and
a treatment solution applied to the pad for contact with the patient's skin, wherein the treatment solution comprises a 0.1% to 20% solution of a proteolytic enzyme.

2. The abrasive pad of claim 1, wherein the treatment solution comprises a 0.5% to 5% solution of a proteolytic enzyme.

3. The abrasive pad of claim 2, wherein the treatment solution comprises a 2% solution of a proteolytic enzyme.

4. The abrasive pad of claim 1, wherein the proteolytic enzyme is papain.

5. The abrasive pad of claim 1, wherein the treatment solution comprises a surfactant.

6. The abrasive pad of claim 5, wherein the surfactant is soap.

7. The abrasive pad of claim 1, wherein the treatment solution contains an antitoxin.

8. The abrasive pad of claim 1, wherein the treatment solution contains an antibiotic.

9. The abrasive pad of claim 1, wherein the treatment solution contains an antifungal treatment.

10. The abrasive pad of claim 1, wherein the treatment solution contains an antiseptic.

11. The abrasive pad of claim 1, wherein the treatment solution contains an antibacterial agent.

12. The abrasive pad of claim 11, wherein the antibacterial agent is trichlosan.

13. The abrasive pad of claim 1, wherein the treatment solution contains a soothing and healing agent.

14. The abrasive pad of claim 11, wherein the soothing and healing agent is aloe.

15. The abrasive pad of claim 1, wherein the treatment solution contains alcohol.

16. The abrasive pad of claim 15, wherein the alcohol is methanol.

17. The abrasive pad of claim 1, wherein the treatment solution is a pH titer.

18. The abrasive pad of claim 1, wherein the pad is multilayered.

19. The abrasive pad of claim 18, wherein the pad comprises an absorbent material carrying said treatment solution, and a porous abrasive material.

20. The abrasive pad of claim 1, wherein the pad comprises a burstable receptacle containing said treatment solution.

21. The abrasive pad of claim 20, wherein the pad comprises three layers including two outer layers and a central layer, at least one of the outer layers being of an abrasive material, and the burstable receptacle forming the central layer.

22. The abrasive pad of claim 20, wherein a plurality of burstable receptacles are provided, at least one of said burstable receptacles containing a different treatment solution from at least one other of said burstable receptacles.

23. The abrasive pad of claim 1, wherein said abrasive and entangling surface is formed of matted non-woven fibers.

24. The abrasive pad of claim 1, wherein said abrasive and entangling surface is formed of a loop fabric.

25. The abrasive pad of claim 1, wherein said abrasive and entangling surface is sponge-like.

26. The abrasive pad of claim 1, wherein the pad is a mitt adapted to be worn over a hand of a user.

27. The abrasive pad of claim 1, wherein the sting has been caused by an arthropod.

28. The abrasive pad of claim 27, wherein the arthropod is Solenopsis Invicta.

29. The abrasive pad of claim 1, wherein the sting was caused by a cnidarian.

30. The abrasive pad of claim 29, wherein the cnidarian is a larval of the thimble jellyfish.
